SEQUENCE LISTING (10 sequences)

SEQ ID NO: 1 - DNA, 1725 bp, Human orthopneumovirus
  source 1..1725: mol_type = genomic DNA
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - protein, 574 aa, Human orthopneumovirus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 897 bp, Human orthopneumovirus
  source 1..897: mol_type = genomic DNA
atgagcaagaacaaggaccagcggaccgccaagaccctggaacggacctgggacaccctgaaccatctgctgttcatcagtagctgcctgtacaagctgaacctgaagtccgtggcccagatcaccctgagcatcctggccatgatcatcagcaccagcctgatcattgccgccatcatctttatcgccagcgccaaccacaaagtgacccccaccacagccatcatccaggacgccacctcccagatcaagaacaccacccccacctacctgacccagaaccctcagctgggcatcagccccagcaaccccagcgagatcaccagccagatcacaaccatcctggcctccaccacccctggcgtgaagtccaccctgcagagcaccaccgtgaaaaccaagaataccaccaccacacagacccagcccagcaagcccaccaccaagcagagacagaacaagcccccctccaagcccaacaacgacttccacttcgaggtgttcaacttcgtgccctgcagcatctgcagcaacaaccccacctgttgggccatctgcaagcggatccccaacaagaagcccggcaagaaaaccacaaccaagcctaccaagaagcctaccctgaaaaccaccaagaaggaccccaagccccagaccaccaagagcaaagaggtgccaaccaccaagcccaccgaggaacccaccatcaacaccaccaagaccaacatcatcaccaccctgctgacctccaacaccaccggcaaccccgagctgacaagccagatggaaaccttccacagcaccagcagcgagggcaaccctagccctagccaggtgtccaccacctccgagtaccccagccagcctagcagcccccccaacacccccagacagtga

SEQ ID NO: 4 - protein, 298 aa, Human orthopneumovirus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 5 - DNA, 585 bp, Human orthopneumovirus
  source 1..585: mol_type = genomic DNA
atgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctga

SEQ ID NO: 6 - protein, 194 aa, Human orthopneumovirus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 7 - DNA, 1176 bp, Human orthopneumovirus
  source 1..1176: mol_type = genomic DNA
atggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttga

SEQ ID NO: 8 - protein, 391 aa, Human orthopneumovirus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 9 - DNA, 1809 bp, synthetic construct
  source 1..1809: mol_type = other DNA
atggccctgagcaaagtgaagctgaacgacaccctgaacaaggaccagctgctgtccagctccaagtacaccatccagagaagcaccggcgacagcatcgacacccccaactacgacgtgcagaagcacatcaataagctgtgcggcatgctgctgatcaccgaggacgccaaccacaagttcaccggcctgatcgggatgctgtacgccatgagccggctgggccgggaggacaccatcaagatcctgcgggacgccggctaccacgtgaaggccaacggcgtggacgtgaccacccaccggcaggacatcaacggcaaagaaatgaagttcgaggtgctgaccctggccagcctgaccaccgagatccagatcaacatcgagatcgagagccggaagtcctacaagaaaatgctgaaagaaatgggcgaggtggcccccgagtacagacacgacagccccgactgcggcatgatcatcctgtgtatcgccgccctggtcatcacaaagctggccgctggcgacagatctggcctgaccgccgtgatcagacgggccaacaacgtgctgaagaacgagatgaagcggtacaagggcctgctgcccaaggatatcgccaacagcttctacgaggtgttcgaaaagcacccccacttcatcgacgtgttcgtgcacttcggcattgcccagagcagcaccagaggcggcagcagagtggagggcatcttcgccggcctgttcatgaacgcctacggcgctggccaggtcatgctgagatggggcgtgctggccaagagcgtgaagaacatcatgctgggccacgccagcgtgcaggccgagatggaacaggtggtggaggtgtacgagtacgcccagaagctgggcggcgaggccggcttctaccacatcctgaacaaccccaaggcctccctgctgtccctgacccagttcccccactttagcagcgtggtgctcggaaatgcagccggactgggcatcatgggcgagtaccgcggcacccccagaaaccaggacctgtacgacgccgccaaggcctacgccgagcagctgaaagaaaacggcgtgatcaactacagcgtgctggacctgacagccgaggaactggaagccattaagcaccagctgaaccctaaggacaacgacgtggagctgctgaacttcgatctgctgaaactggccggcgacgtggaaagcaaccctggccccagcagacggaacccctgcaagttcgagatccggggccactgcctgaacggcaagcggtgccacttcagccacaactacttcgagtggccccctcatgctctgctggtccggcagaactttatgctgaaccggatcctgaagtccatggacaagagcatcgataccctgagcgagatcagcggagccgccgaactggatagaaccgaggaatacgccctgggcgtggtcggagtgctggaaagctacatcggcagcatcaacaacatcaccaagcagagcgcctgcgtggccatgagcaagctgctgaccgagctgaacagcgacgatatcaagaagctgcgcgacaacgaagaactgaactcccccaagatccgggtgtacaacacagtgatcagctacattgagagcaaccggaagaacaacaagcagaccatccatctgctgaagcggctgcccgccgacgtgctgaaaaagaccatcaagaacaccctggacatccacaagtccatcaccatcaataaccccaaagaaagcaccgtgtccgacaccaacgaccacgccaagaacaacgacaccacctga

SEQ ID NO: 10 - protein, 602 aa, synthetic construct
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVELLNFDLLKLAGDVESNPGPSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

(19) United States
(12) Patent Application Publication
(10) Pub. No.: US 2026/0248906 A1
(43) Pub. Date: Aug. 27, 2026

(54) Methods of Isolating Poxviruses from Avian Cell Cultures

(71) Applicant: Bavarian Nordic A/S, Hellerup (DK)

(72) Inventors: Susan Hoffmann Thrane, Birkerod (DK); Janus Falhof, Kobenhavn (DK); Henrik Hertz, Herlev (DK); Athina Andrea, Kobenhavn (DK); Markus Kalla, Penzberg (DE)

(73) Assignee: Bavarian Nordic A/S, Hellerup (DK)

(21) Appl. No.: 19/159,394

(22) PCT Filed: Mar. 7, 2024
(86) PCT No.: PCT/EP2024/056010

§ 371 Date: Aug. 25, 2025

(30) Foreign Application Priority Data
Mar. 10, 2023 (EP) 23161203.7

(51) Int. Cl. A61K 39/155; A61K 39/00; A61K 39/285; C12N 5/071; C12N 7/00; C12N 9/22; C12N 15/86

(52) U.S. Cl. CPC A61K 39/155; A61K 39/285; C12N 5/0602; C12N 7/00; C12N 9/22; C12N 15/86; A61K 2039/5256; A61K 2039/545; A61K 2039/70; C12N 2710/24134; C12N 2710/24143; C12N 2710/24151; C12N 2760/18534; C12Y 301/30002

(57) ABSTRACT

The present invention relates to methods of producing poxvirus viral vector-based vaccine products from avian cell lines. In some embodiments, the avian cells are suspension quail cell lines. Pharmaceutical compositions such as vaccines produced by the methods of the invention are also provided. In some embodiments, the poxvirus viral vector is Modified Vaccinia Virus Ankara (“MVA”) or recombinant MVA. In some embodiments, the recombinant MVA encodes heterologous antigens and can be used to produce a vaccine against the antigens. In some embodiments, the recombinant MVA encodes antigens of Respiratory Syncytial Virus (RSV) and the avian cells are used to produce a vaccine against RSV comprising the recombinant MVA and/or the encoded antigens.

### FIELD OF THE INVENTION

The present invention relates to methods of optimizing production of poxvirus viral vector-based vaccine products from avian cell cultures. The poxvirus viral vector can be a Modified vaccinia virus Ankara (“MVA”) or recombinant MVA that encodes heterologous antigens. The viruses from the cell cultures can be used to produce vaccines.

### BACKGROUND OF THE INVENTION

Poxviruses have a long history of providing vaccines for immune protection against infection and disease. One example is Modified Vaccinia Virus Ankara (“MVA”), a highly attenuated strain of vaccinia virus (genus Orthopoxvirus). MVA-BN® virus developed by Bavarian Nordic® A/S has been used as a vaccine against smallpox and monkeypox marketed under the brand names IMVAMUNE®, IMVANEX®, and JYNNEOS®. Moreover, recombinant MVA-BN® virus encoding various heterologous antigens has also been used as a vaccine. For example, a recombinant MVA-BN® virus encoding antigens from four different filoviruses (Ebola virus, Sudan virus, Tai Forest virus, and Marburg virus) provides an improved vaccine against Ebola virus and is disclosed, for example, in WO 2016/034678. This MVABEAR vaccine, used in combination with Zabdeno® vaccine as part of a 2-component vaccine regimen, has been approved for use in the prevention of Ebola virus disease.

The cells approved for production of MVA-BN® virus were primary chicken embryonic fibroblast cells (“CEF” cells). CEF cells have been widely used to study the interactions between cells and viruses and in the production of vaccines. However, there are several drawbacks of primary CEF cells in this context, for example, the time, cost, and labor involved in preparing these cells (see, e.g., Farzaneh et al. (2017) British Poultry Science 58:681-686), variability of the cell substrate in each batch, and the preparation procedure being prone to contamination.

One alternative to CEF cells may be the duck embryo-derived EB66® cell line (see, e.g., Leon et al. (2016) Vaccine 34:5878-85). Continuous avian cell lines have also been developed from the Muscovy duck (Jordan et al. (2016) Avian Pathology 45:137-155) and the peacock (Wang et al. (2022) Poultry Sci. 101:102147). Another alternative to CEF cells may be continuous quail cell lines, which have been produced by various means (see, e.g., Kraus et al. (2011) BMC Proceedings 5 (Suppl. 8): P52; Lee et al. (2008) J. Virol. Meth. 153:22-8). Quail cells lack most of the endogenous retroviral (ERV) sequences detectable in chicken cells. For example, sequences from the subgroup of the endogenous avian retrovirus family termed EAV-HP were found in chickens but are completely absent in quails (see, e.g., Smith et al. ((1999) J. Gen. Virol. 80:261-268). A comparative mapping of quail and chicken genomes revealed that only 393 intact ERV were identified in quail, versus 1212 in chicken (Morris et al. ((2020) BMC Biol. 18:14).

However, manufacturing costs for vaccines remain high, in part because cell lines often fail to meet the desired yields (see, e.g., Hoeksema et al. (2018) Vaccine 36:2093-2103), or because the resulting product has unacceptably high levels of impurities such as total protein and host cell DNA. Regulatory authorities require very low levels of host cell DNA in the final vaccine product, for example, less than 100 nanograms per vaccine dose. Therefore, there remains a need for safe, efficient, and commercially viable methods for processing virus-infected cell lines to produce vaccines, including vaccines comprising recombinant poxviruses.

### BRIEF SUMMARY OF THE INVENTION

The present invention relates to methods of cultivating and processing avian cells infected with poxviruses to produce viral vector-based vaccine products. In some embodiments, the viral vector is Modified Vaccinia Virus Ankara (“MVA”), such as MVA-BN® virus. In some embodiments, the viral vector is a recombinant MVA encoding one or more heterologous antigens and the avian cells are used to produce a vaccine comprising the recombinant MVA and/or the encoded antigens. In some embodiments, the recombinant MVA encodes one or more antigens of Respiratory Syncytial Virus (RSV) and the avian cells are used to produce a vaccine protecting against disease caused by RSV. In some embodiments, the recombinant MVA encodes a tumor-associated antigen and the avian cells are used to produce a vaccine that stimulates an immune response to the antigen. In some embodiments, the avian cells are quail cells. Vaccines comprising MVAs and recombinant MVAs are produced by the methods of the invention and thus are also provided by the invention.

### BRIEF DESCRIPTION OF THE DRAWINGS

FIG. 1 shows the effects of Denarase® enzyme addition on the Host Cell DNA (“HCD”) present in collected material (i.e., material collected from a poxvirus-infected cell culture) and the reduction of HCD levels at different steps in the downstream process depicted on the X-axis (see Example 1). HCD content per vaccine dose is shown on the Y axis; note log scale. Each line connects data points from a particular run of the downstream process. Abbreviations on horizontal axis represent stages of the downstream process as follows: PI=post infection with poxvirus (Day 3); PT=post treatment with trypsin; PN=post addition of NaCl; PL=post cell lysis; PF=post depth filtration (5 μM); TF1=sample following TFF filtration; PD-sample following second treatment with Denarase® enzyme; BDS=sample of final product (“Bulk Drug Substance”). The data demonstrate that Denerase® enzyme acts to decrease HCD even in the presence of high salt (1M NaCl).

FIG. 2 shows the effect of NaCl addition on Host Cell DNA (“HCD”) reduction during later steps of the downstream purification process (see Example 1; note log scale). Abbreviations on horizontal axis represent stages of the downstream process as follows: PI=post infection with poxvirus; PT=post treatment with trypsin; PN=post addition of NaCl; PL=post cell lysis; PF=post depth filtration (5 μM); TF=sample following TFF filtration; PD=post treatment with Denarase® enzyme; TF2 UF-sample following second TFF ultrafiltration step; TF2 DF=sample following second TFF diafiltration step; BDS=sample of final product (“Bulk Drug Substance”). These data show the surprising result that NaCl addition produces a lower HCD level in later steps of the process, despite showing an initial increase in earlier steps.

FIG. 3 shows a schematic of the hypothesized effect of the first enzymatic treatment step with trypsin enzymatic activity and nuclease (e.g., TrypLE and Denarase® enzyme) on the harvested cells, amplified virus, and impurities that result from lysis of the cells during the virus purification process (showing MVA-BN-RSV as an exemplary virus). While the invention is not bound by any particular mechanism of operation, it is thought that addition of these enzymatic activities prior to cell disruption facilitates the removal of impurities by decreasing cell clumping so that enzymes are better able to access their substrates, providing the improved virus yield seen from this process. This anti-clumping effect can also be seen in the Transmission Electron micrographs in FIG. 3, showing cell material without added trypsin (TrypLE) (left micrograph) and with added trypsin (TrypLE) (right micrograph). These micrographs demonstrate that the added trypsin decreases the formation of large clumps of cells that are likely to be difficult for enzymes to penetrate. FIG. 3 also demonstrates that the protease having trypsin activity functions in this process to de-aggregate these cells that have been grown in suspension, in contrast to the more common use of trypsin to detach cells grown as an adherent cell culture (e.g., cells growing in a monolayer in a cell culture dish)).

### DETAILED DESCRIPTION OF THE INVENTION

The invention relates to methods of producing viral vector-based vaccine products from avian cell cultures. In some embodiments, the viral vector is Modified Vaccinia Virus Ankara (“MVA”), such as MVA-BN® virus. In some embodiments, the viral vector is a recombinant MVA encoding one or more heterologous antigens and the methods are used to produce a vaccine comprising the recombinant MVA. In some embodiments, the recombinant MVA encodes, for example, one or more antigens of Respiratory Syncytial Virus (RSV) and the methods are used to produce a vaccine comprising the recombinant MVA that protects against RSV-induced lower respiratory tract disease. In some embodiments, the recombinant MVA encodes a Tumor-Associated Antigen and can stimulate an immune response against the antigen. In some embodiments, the avian cells are quail cells from a suspension cell line and are processed according to the methods of the invention to provide a vaccine comprising MVA or recombinant MVA. In some embodiments, the quail cells are CCX.E10 cells (Nuvonis (Vienna, Austria)).

The invention provides methods of processing avian cell lines infected with MVA and recombinant MVA to produce vaccines. The methods comprise a “downstream process” for isolating and purifying the virus from cultures of the cell lines for use in a vaccine. Vaccines comprising MVAs (including MVA-BN® virus) and recombinant MVAs can be produced using the methods of the invention and therefore are also provided by the invention.

The use of vaccinia viruses to protect humans against smallpox has a long history, and includes the use of the chorioallantois vaccinia virus Ankara (CVA) that was maintained in the Vaccination Institute in Ankara, Turkey, for many years. However, there were often severe post-vaccine complications associated with these viruses, so efforts were made to generate a more attenuated, safer smallpox vaccine. The attenuated CVA-derived virus Modified Vaccinia Virus Ankara (“MVA”) was obtained by serial propagation of more than 570 passages of CVA on primary chicken embryo fibroblasts (“CEF” cells; for review see Mayr et al. (1975) Infection 3:6-14). As a result of the passaging used to attenuate MVA, there are a number of different strains or isolates of MVA, which are sometimes named according to the passage number in CEF cells. For example, MVA-572 was used in Germany during the smallpox eradication program, and MVA-575 was extensively used as a veterinary vaccine. MVA-575 was deposited on Dec. 7, 2000, at the European Collection of Animal Cell Cultures (ECACC) as deposit number V00120707.

### MVAs and Recombinant MVAs

Strains of MVA having enhanced safety profiles for the development of safer products, such as vaccines or pharmaceuticals, have been developed by Bavarian Nordic® A/S. For example, MVA was further passaged by Bavarian Nordic® A/S and is designated MVA-BN® virus. MVA as well as MVA-BN® virus lacks approximately 15% of the genome compared with the ancestral CVA virus; specifically, 31 kb from six regions. These deletions affect a number of virulence and host range genes, as well as the gene for Type A inclusion bodies. A sample of MVA-BN® virus corresponding to passage 583 was deposited on Aug. 30, 2000 at the European Collection of Cell Cultures (ECACC) under deposit number V00083008.

MVA-BN® virus can attach to and enter human cells, where virally-encoded genes are expressed very efficiently. However, assembly and release of progeny virus does not occur, and no infectious virus is produced. Although MVA-BN® virus does not replicate in human cells, it is strongly adapted to primary chicken embryo fibroblast (CEF) cells. MVA-BN® virus is classified as a Biosafety Level 1 organism according to the Centers for Disease Control and Prevention in the United States. Preparations of MVA-BN® virus and derivatives have been administered to many types of animals and to more than 2000 human subjects, including immune-deficient individuals. All vaccinations have proven to be generally safe and well tolerated. Despite its high attenuation and reduced virulence, in preclinical studies, MVA-BN® virus has been shown to elicit both humoral and cellular immune responses to vaccinia and to heterologous gene products encoded by genes cloned into the MVA genome (see Harrer et al. (2005) Antivir. Ther. 10 (2): 285-300; Cosma et al. (2003) Vaccine 22 (1): 21-9; Di Nicola et al. (2003) Hum. Gene Ther. 14 (14): 1347-1360; and Di Nicola et al. (2004) Clin. Cancer Res. 10(16): 5381-5390).

The virus MVA-BN® is licensed under the tradename IMVANEX® in the European Union (EU) for the prevention of smallpox infection, and under IMVAMUNE® in Canada for the prevention of smallpox, monkeypox and other Orthopoxvirus infections. In the United States, MVA-BN® virus is licensed under the trade name JYNNEOS™ for active immunization against smallpox and monkeypox in adults considered at high risk for these diseases. Thus, MVA-BN® virus itself (i.e., not expressing any additional heterologous antigens) is also a vaccine that can be produced using the methods of the invention.

“Derivatives” or “variants” of MVAs are viruses exhibiting essentially the same replication characteristics as MVA as described herein but have differences in one or more parts of their genomes. MVA-BN® virus as well as a derivative or variant thereof fails to reproductively replicate in vivo in humans and mice, even in severely immune-suppressed mice. More specifically, MVA-BN® virus or a derivative or variant thereof preferably also has the capability of reproductive replication in chicken embryo fibroblasts (CEF), but no capability of reproductive replication in the human keratinocyte cell line HaCat (Boukamp et al. (1988) J. Cell Biol. 106:761-771), the human bone osteosarcoma cell line 143B (ECACC No. 91112502), the human embryo kidney cell line 293 (ECACC No. 85120602), and the human cervix adenocarcinoma cell line HeLa (ATCC No. CCL-2). Additionally, a derivative or variant of MVA-BNR virus has a virus amplification ratio at least two-fold less and more preferably three-fold less than MVA-575 in Hela cells and HaCaT cell lines. Tests and assays for these properties of MVA variants are described in WO 2002/042480 (US 2003/0206926) and WO 2003/048184 (US 2006/0159699). The amplification or replication of a virus is normally expressed as the ratio of virus produced from an infected cell (output) to the amount originally used to infect the cell (input), referred to as the “amplification ratio.” An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1 (i.e., a decrease in output compared to the input level) indicates a lack of reproductive replication and therefore attenuation of the virus.

The advantages of MVA-based vaccines include their safety profile as well as availability for large scale vaccine production. Preclinical tests have revealed that MVA-BN® virus demonstrates superior attenuation and efficacy compared to other MVA strains (WO 2002/042480). An additional property of MVA-BN® virus strains is the ability to induce substantially the same level of immunity in prime/boost regimes utilizing vaccinia virus prime and vaccinia virus boost when compared to regimes utilizing a DNA prime and vaccinia virus boost. The recombinant MVA-BN® viruses, the most preferred embodiment herein, are considered to be safe because of their distinct replication deficiency in mammalian cells and their well-established avirulence. Also, with MVA-BN® virus, the feasibility of industrial scale manufacturing can be beneficial. Furthermore, MVA-based vaccines can deliver multiple heterologous antigens and allow for simultaneous induction of humoral and cellular immunity.

In addition to recombinant MVAs expressing RSV antigens (referred to herein generally as “MVA-RSV”), discussed in detail below, other recombinant MVAs can be produced using the methods of the invention comprising culture in quail cells. Any recombinant MVA that can reproduce in quail cells can be produced using the methods of the invention. Accordingly, the invention provides methods of preparing vaccines comprising any recombinant MVA that can reproduce in quail cells. Such recombinant MVAs include, for example, those expressing EBV antigens (“MVA-EBV”), Equine Encephalitis Virus antigens, Foot and Mouth Disease Virus antigens, filovirus antigens, as well as other disease, viral, or cancer-related antigens or tumor-associated antigens, or heterologous genes such as monomeric Red Fluorescent Protein (mRFP; see, e.g., Campbell et al. (2002) Proc. Nat'l. Acad. Sci. USA 99:7877-82). Thus, it will be appreciated that recombinant MVAs for use in the methods and compositions of the invention comprise heterologous nucleotide sequences encoding one or more heterologous antigens.

Since the MVAs and recombinant MVA viruses described herein are highly replication restricted and thus highly attenuated, they are ideal candidates for the treatment of a wide range of mammals including humans and even immune-compromised humans. MVAs and recombinant MVAs produced in the methods, cells, cell cultures, and populations of cells of the invention can be isolated and/or purified and used to provide compositions for further use, including pharmaceutical compositions such as vaccines. Suitable techniques and formulations for these purposes are known in the art.

In another aspect, an MVA viral strain suitable for generating the recombinant virus may be strain MVA-572, MVA-575, or any similarly attenuated MVA strain. Also suitable may be a mutant MVA, such as the deleted chorioallantois vaccinia virus Ankara (dCVA). A dCVA comprises del I, del II, del III, del IV, del V, and del VI deletion sites of the MVA genome. The deletion sites are particularly useful for the insertion of multiple heterologous sequences. The dCVA can reproductively replicate (with an amplification ratio of greater than 10) in a human cell line (such as human 293, 143B, and MRC-5 cell lines), which then enables optimization by further mutation and can be useful for a virus-based vaccination strategy (see WO 2011/092029).

### MVA-RSV

RSV is a significant respiratory pathogen and is the most clinically important cause of acute lower respiratory tract (LRT) infection, which causes significant morbidity and mortality in infants and children under the age of five years worldwide (see, e.g., Aliyu et al. (2010), Bayero J. Pure Appl. Sci. 3 (1): 147-155). Primary infection with RSV does not induce complete immunity to RSV, so frequent re-infections occur throughout life, with the most severe infections developing in the very young, the very old, and in immune-compromised patients of any age (see, e.g., Murata (2009) Clin. Lab. Med. 29 (4): 725-39).

RSV is an enveloped RNA virus of the family Paramyxoviridae. Each RSV virion contains a non-segmented, negative-sense, single-stranded RNA molecule containing ten genes encoding eleven separate proteins, including eight structural (G, F, SH, M1, N, P, M2-1, and L) and three non-structural proteins (NS1, NS2, and M2.2); M2 contains two open reading frames (Murata (2009) Clin. Lab. Med. 29 (4): 725-39). Much is known about the roles and interactions of RSV proteins.

Recombinant MVAs expressing at least one RSV antigen are referred to herein generally as MVA-RSV. Some embodiments of an MVA-RSV comprise MVA-BN® virus and are referred to herein generally as MVA-BN-RSV.

It had been shown that vaccination with a recombinant vaccinia virus Ankara (MVA) expressing at least one antigen of an RSV membrane glycoprotein and at least one antigen of an RSV nucleocapsid protein (e.g., MVA-mBN201B) induced better immune protection than an RSV vaccine comprising only the RSV-F and/or RSV-G antigens (see WO 2014/019718). In addition, such constructs induced almost complete sterile immunity when applied by the intranasal route, and enhanced protection could be obtained by administering candidate RSV vaccines intranasally in comparison to intramuscular or subcutaneous administration (see, e.g., Wyatt et al. (2000) Vaccine 18:392-97). Unfortunately, in a phase 3 clinical trial conducted by Bavarian Nordic A/S, this recombinant MVA did not meet all the primary endpoints of preventing lower respiratory tract disease from RSV and commercial development of this vaccine candidate was discontinued (see Bavarian Nordic A/S press release dated Jul. 22, 2023).

### RSV Nucleotide Sequences and Proteins

Exemplary RSV vaccines that can be produced using methods of the instant invention are known in the art, for example, as described in WO 2014019718, incorporated specifically in its entirety herein by reference. For use in the methods of the invention, recombinant MVAs encoding RSV genes (herein, “MVA-RSVs”) as mentioned herein refer to the genes, or to a homolog or variant of the genes, encoding the corresponding protein in any RSV strain or isolate, even though the exact sequence and/or genomic location of the gene may differ between strains or isolates. Likewise, the RSV proteins mentioned herein refer to proteins, or to a homolog or variant of the proteins, encoded and expressed by the corresponding gene as defined above. Generally, MVA-RSVs encode RSV proteins that are antigens. In some embodiments, an MVA-RSV is an “MVA-BN-RSV” that comprises MVA-BN® virus such as, for example, MVA-mBN294B.

When referring to the RSV F protein gene, other terms may also be used, such as “F protein gene,” “F glycoprotein gene,” “RSV F glycoprotein gene,” or “F gene,” all of which refer to the gene, or to a homolog or variant of the gene, encoding the transmembrane fusion glycoprotein in any RSV strain or isolate, even though the exact sequence and/or genomic location of the F protein gene may differ between RSV strains or isolates. For example, in the A2 strain of RSV, the F (A2) protein gene comprises nucleotides 5601-7499 (endpoints included) as numbered in GenBank Accession Number M11486. The F (A2) protein gene further comprises a protein coding open reading frame (ORF) spanning nucleotides 5614-7338 (endpoints included) as numbered in GenBank Accession No. M11486. The nucleotide sequence of the F protein gene from RSV A2 (SEQ ID NO:1) is known in the art (see WO 2014019718).

Also interchangeably used herein are the terms “F protein,” “F glycoprotein,” “RSV F protein,” “RSV F glycoprotein,” or “F” which refer to the heavily glycosylated transmembrane fusion glycoprotein, or to a homolog or variant of the protein encoded and expressed by an RSV F protein gene as defined above. The amino acid sequence of the F protein from RSV A2 (SEQ ID NO: 2) is known in the art (see WO 2014019718). The RSV (A2) F protein comprises a signal peptide, an extracellular domain, a transmembrane domain, and a cytoplasmic domain (see, e.g., UniProtKB/Swiss-Prot Accession No. P03420). Other proteins and fragments are known in the art and can be used to provide MVA-RSVs, which in turn can be produced using the methods of the instant invention to provide vaccines.

When referring to the RSV G protein G, similarly, other terms such as “G protein gene,” “G glycoprotein gene,” “RSV G protein gene,” “RSV G glycoprotein gene,” and “G gene” are used interchangeably herein. For example, in the A2 strain of RSV, the G (A2) protein gene comprises nucleotides 4626-5543 (endpoints included) as numbered in GenBank Accession Number M11486. The G (A2) protein gene further comprises a protein coding open reading frame (ORF) spanning nucleotides 4641-5537 (endpoints included) as numbered in GenBank Accession No. M11486. The nucleotide sequence of the G protein gene from RSV A2 (SEQ ID NO: 3) is known in the art (see WO 2014019718).

The terms “G protein,” “G glycoprotein,” “RSV G protein,” “RSV G glycoprotein,” or “G” refer to the heavily glycosylated transmembrane attachment glycoprotein, or to a homolog or variant of the protein. The amino acid sequence of the G protein from RSV A2 (SEQ ID NO: 4) is known in the art (see WO 2014019718). RSV A2 G protein comprises an extracellular domain, a transmembrane domain, and a cytoplasmic domain (see, e.g., UniProtKB/Swiss-Prot Accession No. P03423). These domains are also known in the art and taught in WO 2014019718; for example, the extracellular domain of RSV A2 G protein consists of amino acids 67-298 of SEQ ID NO:4; the transmembrane domain of RSV A2 G protein consists of amino acids 38-66 of SEQ ID NO:4; and the cytoplasmic domain of RSV A2 G protein consists of amino acids 1-37 of SEQ ID NO:4 of WO 2014019718.

Interchangeably used herein are also the terms “M2-1 protein gene,” “M2.1 protein gene,” “M2-1 transcription elongation factor,” “RSV M2-1 protein gene,” “RSV M2.1 protein gene,” “RSV M2-1 transcription elongation factor gene,” or “M2 gene.” For example, in the A2 strain of RSV, the M2 (A2) protein gene comprises nucleotides 7550-8506 (endpoints included) as numbered in GenBank Accession Number M11486. The M2 (A2) protein gene further comprises a protein coding open reading frame (ORF) spanning nucleotides 7559-8143 (endpoints included) as numbered in GenBank Accession No. M11486. The nucleotide sequence of the M2 protein gene from RSV A2 (SEQ ID NO:5) and the amino acid sequence of the M2 protein from RSV A2 (SEQ ID NO:6) are known in the art (see WO 2014019718; see, e.g., UniProtKB/Swiss-Prot Accession No. P04545).

When referring to the RSV N protein gene, the terms “N protein gene,” “N nucleocapsid protein gene,” “RSV N nucleocapsid protein gene,” or “N gene” may be used interchangeably herein. For example, in the A2 strain of RSV, the N (A2) protein gene comprises nucleotides 1081-2277 (endpoints included) as numbered in GenBank Accession Number M11486. The N (A2) protein gene further comprises a protein coding open reading frame (ORF) spanning nucleotides 1096-2271 (endpoints included) as numbered in GenBank Accession No. M11486. The nucleotide sequence encoding the N protein gene from RSV A2 (SEQ ID NO:7) is known in the art (see WO 2014019718). The amino acid sequence of the RSV N protein (also referred to as “N protein,” “N nucleocapsid protein,” “RSV N nucleocapsid protein,” or “N”) from RSV strain A2 (SEQ ID NO:8) is known in the art (see WO 2014019718; UniProtKB/Swiss-Prot Accession No. P03418).

### Certain Recombinant MVAs Encoding RSV Antigens

Recombinant MVAs used in the methods and compositions of the invention can encode one or more RSV antigens suitable for use in an RSV vaccine (generally referred to herein as “MVA-RSV”). For example, recombinant MVAs for use in an RSV vaccine can comprise at least one nucleotide sequence encoding an antigen of an RSV membrane glycoprotein and at least one nucleotide sequence encoding an antigen of an RSV nucleocapsid protein.

In some embodiments, an MVA-RSV encodes antigens of the surface fusion protein (F), two glycoproteins (G) from RSV subtype A and RSV subtype B, and internal proteins which are the nucleoprotein N and the transcription elongation factor M2-1. In some embodiments, the F protein is based on the native surface protein F from the RSV A subtype.

In certain embodiments, an MVA-RSV encodes at least one antigen of an RSV membrane glycoprotein. In certain embodiments, the MVA-RSV encodes an RSV F antigen and/or an RSV G antigen. In certain embodiments, the recombinant MVA encodes an RSV F antigen that is derived from RSV strain A2 and/or an RSV G antigen that is derived from RSV strain A2. In certain embodiments, an MVA-RSV encodes at least two antigens of an RSV membrane glycoprotein that are an RSV F antigen and an RSV G antigen. In certain embodiments, the RSV F antigen and/or the RSV G antigen are derived from RSV strain A2.

In certain embodiments, the MVA-RSV encodes an antigen of an RSV membrane glycoprotein and comprises at least one heterologous nucleotide sequence encoding an antigen of an RSV nucleocapsid protein. In certain embodiments, the recombinant MVA encodes at least one antigen of an RSV F membrane glycoprotein and at least one antigen of an RSV M2 nucleocapsid protein or an RSV N nucleocapsid protein. In certain embodiments, the MVA-RSV encodes at least an antigen of an RSV G membrane glycoprotein and an antigen of an RSV M2 nucleocapsid protein and/or an antigen of an RSV N nucleocapsid protein.

In certain embodiments, the MVA-RSV encodes at least one antigen of each of: an RSV F membrane glycoprotein; an RSV G membrane glycoprotein; and an RSV M2 or N nucleocapsid protein. In certain embodiments, both the RSV F antigen and the RSV G antigen are derived from RSV strain A2. In certain embodiments, the MVA-RSV encodes at least one antigen of each of: an RSV F membrane glycoprotein, an RSV G membrane glycoprotein, an RSV M2 nucleocapsid protein, and an RSV N nucleocapsid protein. In certain embodiments, both the RSV F antigen and the RSV G antigen are derived from RSV strain A2.

In certain embodiments, an MVA-RSV that encodes an antigen of an RSV F membrane glycoprotein encodes a full-length RSV F membrane glycoprotein or alternatively encodes a truncated or partial RSV F membrane glycoprotein, and/or is a variant of the wildtype RSV F membrane glycoprotein. In certain embodiments, the MVA-RSV encodes a full-length RSV F membrane glycoprotein from strain A2, or a full-length, truncated, or variant RSV F antigen derived from RSV strain A_{Long}. In some embodiments, the MVA-RSV encodes a truncated RSV (A_{Long}) F antigen and/or RSV (A2) F antigen that lacks the cytoplasmic and transmembrane domains of the native F protein.

In certain embodiments, MVA-RSV encodes an antigen of an RSV G membrane glycoprotein, optionally from RSV strain A2 or B, which is full-length or truncated, or a variant of wildtype RSV G protein. In certain embodiments, the MVA-RSV encodes a truncated RSV G antigen that lacks the cytoplasmic and transmembrane domains of the full-length RSV G protein.

In certain embodiments, the MVA-RSV encodes an antigen of an RSV M2 nucleocapsid protein which is full-length, truncated, or a variant of wildtype RSV M2 protein, and in some embodiments is derived from RSV strain A2. In certain embodiments, the MVA-RSV encodes an antigen of an RSV N nucleocapsid protein which is full-length, truncated, or a variant of wildtype RSV N protein, and in some embodiments is derived from RSV strain A2. In certain embodiments, the MVA-RSV encodes an antigen of RSV N and an antigen of RSV M2 that are encoded by a single open reading frame and separated by a self-cleaving protease domain, for example such as the self-cleaving protease 2A fragment from Foot and Mouth Disease Virus. In certain embodiments, the MVA-RSV comprises a heterologous nucleotide sequence encoding an RSV N antigen and an RSV M2 antigen that comprises the nucleotide sequence set forth in SEQ ID NO: 9 and/or that encodes the amino acid sequence of SEQ ID NO:10.

In certain embodiments (referred to herein for convenience as “Embodiment A”), the recombinant Modified Vaccinia Virus Ankara (MVA) comprises: (a) at least one nucleotide sequence encoding an antigen of a respiratory syncytial virus (RSV) membrane glycoprotein, wherein the nucleotide sequence encodes a full-length RSV F membrane glycoprotein; and (b) at least one nucleotide sequence encoding RSV nucleocapsid antigens, wherein the nucleotide sequence encodes both a full-length RSV N nucleocapsid protein and a full-length RSV M2-1 transcription elongation factor protein, which are encoded by a single open reading frame, wherein the single open reading frame comprises a nucleotide sequence encoding the amino acid sequence of SEQ ID NO:12 or comprises the nucleotide sequence of SEQ ID NO:11; and further comprises (c) at least one nucleotide sequence encoding an antigen of an RSV G membrane glycoprotein, wherein the nucleotide sequence encodes a full-length RSV G membrane glycoprotein.

Embodiment (B) is the recombinant MVA of embodiment (A), wherein the nucleotide sequence encoding the RSV F membrane glycoprotein is from RSV strain A, preferably from A2 and/or A_{Long}. Embodiment (C) is the recombinant MVA of embodiment (A) or embodiment (B), wherein the nucleotide sequence encoding the RSV F membrane glycoprotein comprises a nucleotide sequence encoding the amino acid sequence of SEQ ID NO:2 or comprises the nucleotide sequence of SEQ ID NO:1. Embodiment (D) is the recombinant MVA of any of Embodiments (A), (B), or (C), wherein the nucleotide sequence encoding the RSV G membrane glycoprotein is from RSV strain A, preferably from strain A2 and/or B. Embodiment (E) is the recombinant MVA of Embodiment (A), (B), (C), or (D), wherein the nucleotide sequence encoding the RSV G membrane glycoprotein comprises a nucleotide sequence encoding the amino acid sequence of SEQ ID NO:4 or comprising the nucleotide sequence nucleotide sequence of SEQ ID NO:3.

Embodiment (F) is the recombinant MVA of embodiment (A), (B), (C), (D), or (E), wherein the MVA used for generating the recombinant MVA is MVA-BN® virus deposited at the European Collection of Cell Cultures (ECACC) under number V00083008. Embodiment (G) is the recombinant MVA of embodiment (A), (B), (C), (D), (E), or (F) for use in preventing at least one symptom of RSV infection, or in preventing RSV-caused disease. Embodiment (H) is the recombinant MVA of embodiment (G) for use in preventing at least one symptom of RSV infection or in preventing RSV-caused disease, wherein the recombinant MVA is administered intranasally and/or subcutaneously, preferably intranasally.

In some embodiments, the recombinant MVA (MVA-RSV) for use in the methods and compositions of the invention is an MVA-BN-RSV, for example, MVA-mBN294B (see, e.g., WO 2014019718, specifically incorporated herein by reference). Several clinical trials of MVA-mBN294B were conducted and successfully demonstrated the safety and immunogenicity of this vaccine. These trials include the Phase 1 trial RSV-MVA-001 and the Phase 2 trial RSV-MVA-002, which provided promising safety and immunogenicity results. Unfortunately a Phase 3 clinical trial of this RSV vaccine candidate for adults 60 years of age and older did not meet all the primary endpoints of preventing lower respiratory tract disease (LRTD) caused by RSV, and the commercial development of this vaccine candidate was discontinued (see Bavarian Nordic A/S press release dated Jul. 22, 2023).

Immunogenicity was assessed by evaluating antibody and T cell responses, and doses of vaccine were administered at 1×10⁸ Infectious Units (IU) per 0.5 mL or 5×10⁸ IU per 0.5 mL. The results of the completed Phase 1 and 2 trials showed that a single dose of MVA-BN-RSV induced broad humoral, cellular and mucosal immune responses persisting over at least 6 months. A single dose of MVA-BN-RSV vaccine elicited increases in neutralizing antibodies (identified using PRNT to RSV-A and B subtype) and total antibodies (IgG and IgA ELISA) as well as a broad Th1-biased cellular immune response (IFN-γ/IL-4 ELISPOT) to all 5 inserts encoded in the vaccine also confirming results from non-clinical studies in different animal models. Overall, the vaccine-induced antibody responses (Geometric Mean Titers (GMT)) remained above baseline 30 weeks post-vaccination.

### Vaccines and Pharmaceutical Compositions

Poxviruses such as the MVAs and recombinant MVA viruses described herein are highly replication restricted and, thus, highly attenuated, they are ideal candidates for the treatment of a wide range of mammals including humans and even immune-compromised humans. Thus, provided herein are methods for producing these recombinant MVAs and compositions comprising them for use as pharmaceutical compositions and vaccines, all intended for inducing an immune response in a living animal body, including a human.

For this, the MVA or recombinant MVA, vaccine, or pharmaceutical composition can be formulated in solution in a concentration range of about 10⁴ to 10⁹ IU/mL, 10⁵ to 5×10⁸ IU/mL, 10⁶ to 10⁸ IU/mL, or 10⁷ to 10⁸ IU/mL. A preferred dose for humans comprises between 10⁶ to 10⁹ IU/mL, including a dose of at least about: 10⁶ IU/mL, 10⁷ IU/mL, 10⁸ IU/mL or 5×10⁸ IU/mL. In some embodiments, a pharmaceutical composition that is a vaccine comprising MVA or MVA-RSV comprises 1×10⁸ IU/0.5 mL or 5×10⁸ IU/0.5 mL (i.e., 1×10⁸ IU in a volume of 0.5 mL or 5×10⁸ IU in a volume of 0.5 mL), or about 1.58×10⁹ InfU/mL in a final (vaccine) volume of 0.5 mL.

The pharmaceutical compositions produced by the methods of the invention may generally include one or more pharmaceutically acceptable and/or approved buffers, carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, and the like.

For the preparation of vaccines, the poxviruses such as MVAs or recombinant MVAs produced by the methods of the invention can be converted into a physiologically acceptable form. This can be done, for example, based on experience in the preparation of poxvirus vaccines used for vaccination against smallpox as described by Stickl et al. ((1974) Dtsch. med. Wschr. 99:2386-2392). For example, purified viruses can be stored at −80° C. with a titer of 5×10⁸ IU/mL formulated in about 10 mM Tris, 140 mM NaCl pH 7.7. For the preparation of vaccine doses, e.g., 10²-10⁸ or 10²-10⁹ particles of the virus can be lyophilized in 100 ml of phosphate-buffered saline (PBS) in the presence of 2% peptone and 1% human albumin in an ampoule, preferably a glass ampoule. Alternatively, the vaccine doses can be produced by stepwise freeze-drying of the virus in a formulation. This formulation can contain additional additives such as mannitol, dextran, sugar, glycine, lactose, or polyvinylpyrrolidone or other aids such as antioxidants or inert gas, stabilizers or recombinant proteins (e.g., human serum albumin) suitable for in vivo administration. In some embodiments, the glass ampoule is then sealed and can be stored between 4° C. and room temperature for several months. However, the ampoule can also be stored, for example, at temperatures below-20° C.

For administration to a subject, the lyophilisate can be dissolved in an aqueous solution, preferably physiological saline or Tris buffer, and administered either systemically or locally, i.e., by a route of administration that is parenteral, subcutaneous, intravenous, intramuscular, intranasal, or any other suitable path of administration. The mode of administration, the dose, and the number of administrations can be optimized by those skilled in the art in a known manner. However, most commonly a subject is vaccinated with a second shot about one month to six weeks after the first vaccination shot.

In some embodiments, vaccination with an MVA or recombinant MVA of the invention (i.e., a recombinant MVA of the invention or a recombinant MVA produced by the methods of the invention) produces sterile immunity in a treated subject. However, sterile immunity is not required for a vaccine to provide benefit to a vaccinated subject. The usefulness of recombinant MVAs as vaccines is illustrated, for example, by MVA-BN-RSV (i.e., MVA-mBN294B; see data provided in working examples). Vaccination of a subject with MVA-BN-RSV optimally protects against RSV-caused disease by stimulating various aspects of the adaptive immune system such as, for example, the production of RSV-specific antibodies and/or T cells. Efficacy can be assessed using an animal model such as the RSV challenge model in BALB/c mice (see, e.g., Waris et al. (1996) J. Virol. 70:2852-60). Efficacy of stimulation of the immune response can be evaluated, for example, by decrease in the viral load of vaccinated subjects in the event of exposure to RSV and possible subsequent infection. Efficacy can also be assessed across a population of vaccinated subjects, as a statistical decrease in the occurrence or severity of infections, as is known in the art.

In this manner, the methods and compositions of the invention provide vaccines that are useful in protecting subjects against diseases, for example, Lower Respiratory Tract Disease caused by RSV, or smallpox or monkeypox. Other recombinant MVAs or MVAs (such as MVA-BN® virus) produced using the methods of the invention can similarly be evaluated with corresponding assays known in the art. In some embodiments, an MVA or recombinant MVA produced using the methods of the invention stimulates an immune response in a vaccinated subject that increases the production of specific antibodies and/or T cells above a background level observed prior to vaccination.

### Definitions

The term “antigen” as used herein refers to any molecule that stimulates a host's immune system to make an antigen-specific immune response, whether a cellular response and/or a humoral antibody response. Antigens may include proteins, polypeptides, protein fragments, and epitopes that elicit an immune response in a host. Thus, antigens that are proteins, polypeptides, protein fragments, and epitopes are not limited to particular native amino acid sequences but also encompass modifications to the native sequence, such as deletions, additions, insertions, and substitutions that result in variant amino acid sequences. Antigen-encoding sequences may be from another virus or pathogen, or associated with a disease such as cancer or a tumor, or may be another sequence.

By a “day” as used herein is approximately 24 hours, or at least 12 hours but less than 36 hours. By “overnight” as used herein is approximately about 6 or 8 hours, or about 8 to 12 hours, or about 12 to 16 hours.

Preferably, as the term is used herein, “sequence variants” or “variants” have at least about 80% or 85%, or at least about 90%, 91%, 92%, 93%, or 94% or at least about 95%, 96%, 97%, 98% or 99% identity with the referenced nucleic acid or amino acid sequence. The term “variant” also encompasses truncated, deleted, or otherwise modified nucleic acid or protein sequences such as, for example, soluble forms of the RSV-F or RSV-G proteins lacking the signal peptide as well as the transmembrane and/or cytoplasmic domains of the full-length RSV-F or RSV-G proteins and the nucleic acids encoding them, and deleted, truncated, or otherwise mutated versions of the full-length RSV-M2 or RSV-N proteins and the nucleic acids encoding them. A deleted or truncated version of a protein or nucleic acid can differ from the full-length version by deletion or truncation of one or more elements, and/or by deletion or truncation of particular amino acids or nucleic acids, such as fewer than 30, 20, or 10 amino acids or nucleic acids.

Techniques for determining sequence identity between different nucleic acids and between different amino acids are known in the art. Two or more sequences can be compared by determining their “percent identity.” The percent identity of two sequences, whether nucleic acid or amino acid sequences, is the number of exact matches between two aligned sequences divided by the length of the shorter sequences and multiplied by 100. Alternatively, and particularly if it is necessary to introduce gaps in the alignment of sequences to achieve the maximum percent sequence identity, “percent (%) sequence identity” as used herein is the percentage of nucleic acid or amino acid residues in a candidate sequence that are identical with the nucleic acid or amino acid residues in the reference sequence after aligning the sequences and introducing gaps, if necessary, to achieve the maximum percent sequence identity. Alignment for purposes of determining percent amino acid sequence identity can be achieved in various ways that are within the skill in the art, for example, using publicly available computer software such as BLAST, ALIGN, or Megalign (DNASTAR) software. Those skilled in the art can determine appropriate parameters for producing a sequence alignment and calculating sequence identity, including any algorithms needed to achieve maximum alignment over the full length of the sequences being compared. Details of these programs are known in the art and can be found, for example, at the website for the National Center for Biotechnology Information.

As used herein, a “heterologous” gene, nucleic acid, or protein is understood to have a nucleic acid or amino acid sequence which is not present in the wild-type poxviral genome (e.g., sequences encoding antigens of another virus, or pathogen, or associated with a disease such as cancer or a tumor, or another heterologous sequence). The skilled person understands that a nucleic acid which is a “heterologous gene,” when present in a recombinant poxvirus such as MVA, is to be incorporated into the poxviral genome in such a way that, following administration of the recombinant poxvirus to a host cell, it is expressed as the corresponding heterologous gene product, i.e., as the “heterologous antigen” and\or “heterologous protein.” Expression is normally achieved by operatively linking the heterologous gene to regulatory elements that allow expression in the poxvirus-infected cell. In some embodiments, the regulatory elements include a natural or synthetic poxviral promoter.

“Sterile immunity” as used herein means protective immunity provided by a vaccine in the absence of detectable pathogen in a subject (for example, absence of RSV genome when sensitive detection methods, such as RT-qPCR, are applied). By “subject” is intended any animal which is being treated, for example, by administration of a vaccine; as used herein, a subject may be a mammal, including farm animal or companion animal, or may be a human subject or patient.

An “increased inflammatory response” or “enhanced inflammation response” as used herein is characterized by one or more of the following: increased production of IL-12 p70, M-CSF, and/or IL-33; increased antigen-specific CD8+ T cells, increased percentages of CD8+ T cells expressing IFN-gamma and TNF-alpha; decrease in tumor size and/or growth rate; and improved survival of treated subjects, and the like, which can be detected by assays known in the art. As used herein, “increased inflammatory response” generally refers to an increase in production of a particular cytokine or cell type associated with inflammation, in comparison to baseline levels prior to treatment, for example, with compositions of the invention. In an “increased inflammatory response,” the amount of a cytokine or cell type is increased by at least 10%, 20%, 30%, 50%, 70%, or 100% or more in comparison to baseline levels in a subject prior to a treatment.

Viral titer can be measured in several ways; one of skill in the art is familiar with techniques for determining viral titer and comparing different measures of titer. “TCID 50” is the abbreviation of “tissue culture infectious dose,” which is the amount of a pathogenic agent that will produce pathological change in 50% of cell cultures inoculated, expressed as TCID 50/mL. Methods for determining TCID50 are well known to the person skilled in the art, for example, as described in Example 2 of WO 03/053463. “IU” as used herein stands for “Infectious Units” and is also a measurement of viral titer, typically expressed as IU/mL.

Purification of Virus from Avian Cell Cultures

The methods of the invention involve processing avian cells infected with poxviruses to produce viral vector-based vaccine products. Thus, the methods of the invention can be performed using avian cells such as, for example, Chicken Embryonic Fibroblasts (“CEF” cells), duck cells, and quail cells. In some embodiments, the cells are grown in a bioreactor capable of perfusion (i.e., gradual removal of older media and introduction of new media while cells continue to be cultured); alternatively, cells can be grown in cell bags (also referred to as “wave bags”), shaker flasks, spinner flasks, or in any other manner suitable for cell and virus growth.

The cells and/or cell cultures containing the poxvirus such as, for example, MVA or recombinant MVA are cultured to amplify the poxvirus that was used to infect the cells. The cell culture is then harvested and products including viruses can be purified. By “harvested” or “harvesting” as used herein is intended that the cell culture is prepared for further processing to isolate the virus from the cell culture. In some embodiments, this entails centrifugation to separate cells from the cell culture medium, and in some embodiments, the cell culture including both cells and medium is processed to isolate virus, for example, by adding reagents such as salt, nucleases, proteases, and the like; in some embodiments, the cell culture is placed in a suitable tank or vessel for said processing and this consititutes “harvesting.”

The downstream process comprises collection of poxvirus product (e.g., MVA or recombinant MVA) from the cell culture and removal of impurities. In an exemplary embodiment, this process includes steps of: harvesting a cell culture comprising cells infected with MVA or recombinant MVA; treating the cell culture or cells collected therefrom (“material”) with a protease (e.g., trypsin); inactivation of the protease with inhibitor; treatment with a nuclease (e.g., Denarase® endonuclease or Benzonase® endonuclease) and incubating the material; lysing cells in the material (e.g., with high pressure homogenization or ultrasonication); removal of cell debris and remaining intact cells in a first filtration step (e.g., with depth filtration to remove components above 5 μm in size)); continuing incubation under conditions suitable for nuclease activity; and concentrating product (e.g., using tangential flow filtration to retain components larger than 100 nm while removing smaller impurities). The solution or buffer comprising the product can then be changed, if desired, using diafiltration.

In some embodiments of this process, the pH and salt concentration of the material can be adjusted before, after, or at the same time as the addition of the nuclease (e.g., Denarase® endonuclease or Benzonase® endonuclease); in some embodiments, the salt concentration of the material is adjusted to 0.5M NaCl or above, or about 1M NaCl after nuclease is added to the material. In some embodiments, the process further includes a second enzymatic treatment with a nuclease; a second concentration of product (e.g., using tangential flow filtration); and optionally an exchange of buffer with diafiltration. In some embodiments, the product is MVA or recombinant MVA which is provided in a pharmaceutically acceptable buffer or other solution and is thus a pharmaceutical composition. The product can then be stored, for example, at very low temperatures (below −20° C.). The nuclease steps assist with removal of host cell DNA; in some embodiments, the nuclease is Denarase® nuclease, and in other embodiments, any suitable nuclease may be used, such as Benzonase® endonuclease (Sigma Aldrich, St. Louis, MO, USA) or TurboNuclease™ nuclease (VitaScientific, Beltsville, MD, USA). In some embodiments, the product which is an MVA or recombinant MVA can be separated from impurities and other debris using the sucrose cushion technique and/or ultracentrifugation.

By “material” as used herein is intended the remaining matter from the cell culture that is being processed to recover product such as virus, etc.; at various stages, “material” may comprise cells, lysed cells, host cell DNA, residual protein, and the like. The term “product” as used herein refers to the poxvirus (e.g., MVA or recombinant MVA) that was used to infect the cell culture and can be purified by the methods described herein. In some embodiments, when sufficiently purified, the product is suitable for use as a vaccine. By “purified” as used herein is intended that most of the cellular debris and other contaminants such as host cell DNA have been removed from the cell culture material to provide product essentially comprising MVA or recombinant MVA, with little additional contamination or no significant additional contamination, for example, that would interfere with the therapeutic function of the product if used as a vaccine. In some embodiments, product is sufficiently purified for use as a pharmaceutical composition such as a vaccine. For example, as contemplated herein, a product may be considered sufficiently purified for use as a vaccine if the level of host cell DNA is less than 100 ng per vaccine dose (e.g., per dose of 1×10⁸ IU/0.5 mL or 5×10⁸ IU/0.5 mL, or equivalent per dose and volume). That is, a product may be considered sufficiently purified if the level of host cell DNA is less than 100 ng per 0.5 mL volume of vaccine formulation comprising 1×10⁸ IU or 5×10⁸ IU of the virus.

By “about” as used in the context of values of parameters of the methods and compositions of the invention is intended that the actual value of said parameter(s) may vary somewhat from the recited value of said parameter and still have the same qualities and provide the benefit or result of the methods and compositions. Thus, as used herein, a parameter that is recited to have “about” a particular value has an actual value within 10% of the recited value (e.g., a solution having a temperature of “about 30° C.” has a temperature between 27° C. and 33° C.), unless the context clearly indicates otherwise. In this manner, “about” encompasses the recited value of a parameter (e.g., a solution having a temperature of “about 30° C.” can have a temperature of 30° C.).

In an exemplary embodiment(s), the details of the downstream process are as follows: To harvest the cell culture and collect product, material comprising cells and/or virus from the culture is collected into a mixer tank. A first series of enzymatic steps is then performed (sometimes referred to as “Enzymatic Treatment 1”). The material is treated with a protease; in some embodiments, the protease has trypsin activity (e.g., trypsin or recombinant trypsin such as TrypLE (Gibco, Fisher Scientific, Waltham, MA, USA)). In some embodiments, the material is treated with recombinant trypsin (e.g., at a ratio of 1:5) and incubated to allow time for digestion, for example, for 30 minutes to 1 hour at ambient temperature. Before the addition of nuclease, the material is treated to inactivate the protease (e.g., with trypsin inhibitor (1:5) for 15 minutes at ambient temperature) and also to adjust the pH, salt concentration, and volume of the material as needed; these adjustments can be made in any order and can be made simultaneously or separately. For example, the pH of the material is adjusted to about 7.5, 8.0, 8.5, 8.7, or any appropriate pH, for example, any pH in a range between 8.0 and 8.6, such as about 8.0, 8.1, 8.2, 8.3, 8.4, 8.5, 8.6, or 8.7. In some embodiments, MgCl₂ is added to a concentration of about 2 mM or in a range of 2 mM to 25 mM, and a nuclease is added (e.g., Denarase® endonuclease (c-LEcta (Leipzig, Germany)), for example at a concentration of 20 U/mL). NaCl or another salt or chaotropic agent is added to a concentration of about 1M, or at least about 0.5M, 0.6M, 0.7M, 0.8M, 0.9M, 1.0M, 1.1M, 1.2M, 1.3M, or at least about 1.4M. Optionally, the material is cooled to about 4° C., or to between 2° C. and 8° C., for about 2 hours, about 4 hours, about 12 hours, or between 2 and 24 hours, or between 14 and 24 hours while being stirred or agitated, or material can proceed to the next step without incubation (e.g., material can be held at 4° C. overnight; this step can be referred to as “Hold Up 1”). Optionally, if a higher biomass burden is present (i.e., more cells are being treated), the additional impurities and other debris can be removed with minor modifications to this process by increasing the amount of nuclease, increasing the incubation temperature, and/or increasing incubation time during these steps. Also, in some embodiments, the material is diluted as a result of or in addition to the adjustments of pH and salt described above, for example, the volume of the material is increased by about 40%, 50%, 60%, or about 100% or 200%, which may aid in removal of impurities.

Cells are then lysed in order to collect intracellular viruses. This step, which can be referred to as “Cell Lysis,” is accomplished with any suitable technique such as high pressure homogenization (HPH) or ultrasonication, which releases cell debris, including host cell DNA and host cell protein. Impurities including cell debris and remaining cells are then removed, for example, by depth filtration (e.g., through a 5 μm or 3 μm depth filter), a step which can be referred to as “Clarification.” The material can then be stored at ambient temperature or warmed to about 30° C. or in the range between 25-37° C. and incubated overnight or for about 1 hour, or about 2 to 6 hours, or about 8 to 12 hours, or about 22 to 30 hours to allow the nuclease (e.g., Denarase® endonuclease) to degrade the host cell DNA (a step that can be referred to as “Hold Up 2”); in some embodiments, the material is stirred or agitated continually during this step. Optionally, the material is held at </=10° C. This step typically removes much of the host cell DNA.

A further filtration is then performed, for example, using Tangential Flow Filtration (TFF); this step can be referred to as “TFF1.” This step can be performed using any suitable filter, for example, it can be performed with a 0.1 μm PESU E-screen to obtain a concentration (e.g., of 5-6×) via ultrafiltration while removing small impurities, followed with a further concentration (e.g., of 1.3×) using diafiltration (in some embodiments, for a total concentration of at least about 2×, about 4×, about 6×, or about 8×). In some embodiments, for this step, filtration is performed using hollow fiber (HF)-based filtration with a Polysulfone (PS)-based filter with a 0.5 mm diameter and a cutoff of 0.05 μm. In some embodiments, filtration is performed using HF-based filtration with a mixed cellulose ester (“ME”)-based filter with a 0.63 mm diameter and a cutoff of 0.1 μm, and in some embodiments the filter comprises modified polyethersulfone (mPES) with a 0.5 mm diameter and a cutoff of 750 kDa. As described above, this TFF concentrates the product and diafiltration is then performed with diafiltration buffer DF1 (10 mM Tris, 2 mM MgCl₂, pH 8.2), removing impurities and Denarase® endonuclease. Optionally, the system is flushed at the end of this step and the flush pooled with the filtered material in a suitable buffer.

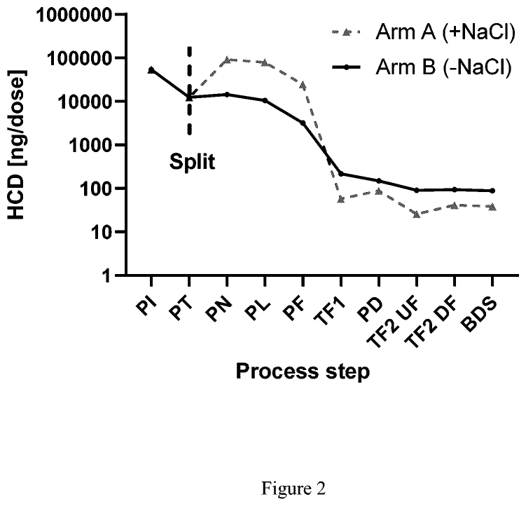

In trial runs, these methods removed large aggregates that were present in the material after cell lysis and provided superior results at later steps and in the final product, including reduced amounts of host cell DNA and total protein. These superior results were unexpected for several reasons, including the recommendation by the Denarase® endonuclease manufacturer (c-LEcta GmbH, Leipzig, Germany) to use this endonuclease at lower salt concentrations to avoid decreasing the efficiency of the enzyme. It was also surprising that addition of relatively high amounts of NaCl early in the process could improve the removal of host cell DNA from the product at a later filtering step (see FIG. 2), and that this effect could not be provided by adding NaCl later in the process. Further, adding an enzyme with trypsin-like activity early in the process also acted to reduce levels of aggregates and improved overall recovery and host cell DNA removal, which was unexpected because trypsin is typically used in upstream processes and could be considered an unnecessary contaminant when used as described here. This downstream process worked well for viral purification from different types of cultures including bioreactor cultures, perfusion bioreactor cultures, and wavebag cultures.

In some embodiments of the downstream process, a second enzymatic treatment is performed with nuclease (e.g., Denarase® endonuclease) to further digest remaining host cell DNA, for example, adding Denarase® endonuclease to a concentration of 75-150 U/mL and incubating overnight (e.g., about 4 to 8 hours, about 8 to 12 hours, or about 14-22 hours) at ambient temperature (~22° C.) or in a range between 25-37° C. If performed, this step can be referred to as “Enzymatic Treatment 2.” In some cases, MgCl₂ is added (e.g., to 2 mM) and pH adjusted to increase nuclease activity; these adjustments can be made before, after, or at the same time as addition of the nuclease to the material. Optionally, the material is held at a low temperature (e.g., less than about 10° C., or less than about 4° C.); this can be referred to as “Hold Up 3.” In some embodiments, the material is continually stirred or agitated during this step to avoid sedimentation and/or aggregation.

In some embodiments, a second filtration step is then performed using Tangential Flow Filtration (TFF), for example, using a 0.1 μm cutoff PESU E-screen (for example, producing a concentration of 14-15× via ultrafiltration (including at least 2 to about 10 exchanges of diafiltration buffer)). Another suitable filter for this step, for example, is an mPES cassette with a 0.01 μm cutoff. This filtration can be performed using diafiltration buffer (DF2) (10 mM Tris, 140 mM NaCl, pH 7.7), and the system can be flushed at the end of this step and the flush pooled with the product, resulting in a final concentration of about 4×. Alternatively, this second filtration step is performed using an HF (hollow fiber)-based TFF filtration step with any suitable filter, for example, PS, 0.5 mm diameter and 0.05 μm cutoff hollow fiber, a mixed cellulose ester (“ME”)-based filter with a 0.63 mm diameter and a cutoff of 0.1 μm, or a modified polyethersulfone (mPES) filter with a 0.5 mm diameter and a cutoff of 750 kDa. First, the material is concentrated and then can be diafiltrated to exchange the buffer. Optionally, the system is flushed at the end of the step and the flush pooled with the filtered material, resulting in a final 10× concentrated product. Product can then be stored, for example, at about −20° C., or at about −80° C.

In some embodiments, during the above process, and particularly in the overnight and/or incubation steps, it is important to avoid sedimentation or settling of the material, as adverse effects on yield may be observed, possibly due to aggregation of virus product with residual debris from the host cells or other viral particles. Accordingly, in some embodiments, it is necessary to stir or otherwise agitate the material during each of the process steps to prevent settling and/or sedimentation, intermittently or continually. In some embodiments, the material is stirred or agitated during steps requiring incubation, such as during an overnight hold, but other steps may be performed without stirring or agitation. The stirring or agitation may be accomplished by any suitable means, for example, using magnetic stirrer bars, shaker platforms, circulation of gas or liquid through the material, and the like.

In some embodiments, the process can be paused or “held up” at any suitable step, or steps can be extended in time, so long as the effect of the step and/or the effect of the overall process are achieved. Thus, while some steps are indicated herein to be “Hold Up” steps, these incubations can be made shorter or longer, or “hold ups” performed at other steps, for convenience or ease of manufacturing.

The methods of the invention provide purification of active virus without the use of chromatography methods, including for example hydrophobic interaction chromatography (“HIC”), and thus have the benefit of not requiring additional materials such as HIC matrix for purification of virus. Thus, the invention provides methods of purification that do not use hydrophobic interaction chromatography or other chromatographic techniques.

In trial runs, results of this process achieved the goals of a high overall virus (product) recovery of at least 50% and with a host cell DNA level below 100 ng per vaccine dose, as preferred. Also, the remaining host cell DNA was fragmented, making any viable coding nucleic acids in the vaccine less likely. These results were superior to those obtained with EB66 duck cells in earlier trials as well as those obtained with CEF cells.

In summary, these methods of producing vaccines comprising poxviruses such as, for example, MVA or recombinant MVA from avian cells provide higher yields and lower impurity levels. These methods also can be used to prepare vaccines comprising poxviruses (for example, MVA or recombinant MVA) from infected quail cell lines, thus making possible the use of cell banks, decreasing the potential for contaminating adventitious agents, and making it possible to manufacture vaccine products without the use of antibiotics. In this manner, use of the methods of the invention in producing poxviruses such as, for example, MVAs and recombinant MVAs can increase the quality and safety of the vaccine comprising these drug substances.

### Certain Embodiments

Certain embodiments of the present invention also include the following items:

Item 1 is a method of processing an avian cell culture or population of cells infected with a poxvirus that is an MVA or recombinant MVA to provide a pharmaceutical composition, comprising the steps of: (a) harvesting cell cultures comprising cells and/or virus, or a population of cells, to produce collected material; (b) treating the collected material with a protease having trypsin activity; (c) optionally, adjusting the pH of the material to 8.0-8.6; (d) treating the material with a nuclease; (e) lysing cells in the material; (f) filtering the material to remove cell debris and remaining cells; (g) continuing incubation with nuclease for a period of time; and (h) concentrating the product using tangential flow filtration. Item 2 is the method of item 1, further comprising the steps of: (i) treating the material with a nuclease; (j) concentrating the MVA or recombinant MVA product using tangential flow filtration. Optionally for each of item 1 and 2 there is a further step of exchanging buffer with diafiltration, and optionally there is a final step of suspending the product in a suitable buffer to provide a pharmaceutical composition.

Item 3 is the method of item 1 or 2 wherein said nuclease in step (d) and/or step (i) is Denarase™ nuclease. Item 4 is the method of item 3, further comprising the addition of NaCl to a final concentration of about 1 M after step (d), during step (d), and/or before step (e). Item 5 is the method of item 3 or 4, wherein step (d) further comprises the addition of NaCl to a final concentration of about 1 M after addition of nuclease; optionally, after NaCl is added, the material is incubated with said nuclease at between 22° C. and 37° C. for at least 2 hours, or between 1 and 24 hours. In some embodiments of Item 3 and Item 4, NaCl is added to a final concentration of at least 0.5M.

Item 6 is a pharmaceutical composition comprising MVA-RSV or MVA-BN-RSV made by the process of any of items 1-5 for the prevention of Lower Respiratory Tract Disease caused by RSV. Item 7 is a pharmaceutical composition comprising an MVA or recombinant MVA made by the method of any of items 1-5, optionally a vaccine containing 100 ng or less of host cell DNA per vaccine dose.

Item 8 is an MVA made by the method of any of items 1-5, or a recombinant modified vaccinia virus Ankara (MVA) comprising a nucleotide sequence encoding an antigen of at least one respiratory syncytial virus (RSV) membrane glycoprotein for treating or preventing disease caused by an RSV infection made by the method of any of items 1-5, optionally in a formulation suitable for intranasal administration.

Item 9 is a recombinant modified vaccinia virus Ankara (MVA) comprising at least one nucleotide sequence encoding an antigen of a respiratory syncytial virus (RSV) membrane glycoprotein and at least one nucleotide sequence encoding an RSV nucleocapsid antigen made by the method of any of items 1-5.

Item 10 is the recombinant MVA of item 8 or 9, wherein the nucleotide sequence encoding an antigen of the RSV membrane glycoprotein encodes an RSV F antigen. Item 11 is the recombinant MVA of item 8 or 9, wherein the nucleotide sequence encoding an antigen of the RSV membrane glycoprotein encodes a full length RSV F membrane glycoprotein. Item 12 is the recombinant MVA of item 10 or 11, wherein the nucleotide sequence encoding an antigen of the RSV F membrane glycoprotein is derived from RSV strain A, preferably from A2 and/or Along.

Item 13 is a recombinant modified vaccinia virus Ankara (MVA) made by the method of any of items 1-5 and comprising: (a) at least one nucleotide sequence encoding an antigen of a respiratory syncytial virus (RSV) membrane glycoprotein, wherein the nucleotide sequence encodes a full-length RSV F membrane glycoprotein; and (b) at least one nucleotide sequence encoding an RSV nucleocapsid antigen, wherein the nucleotide sequence encodes both a full-length RSV N nucleocapsid protein and a full-length RSV M2 matrix protein, which are encoded by a single open reading frame, wherein the single open reading frame comprises a nucleotide sequence encoding the amino acid sequence of SEQ ID NO:10 or comprises the nucleotide sequence of SEQ ID NO:9; and further comprising: (c) at least one nucleotide sequence encoding a full-length RSV G membrane glycoprotein.

Item 14 is the recombinant MVA of item 13 wherein the nucleotide sequence encoding the RSV F membrane glycoprotein is from RSV strain A, preferably from A2 and/or Along. Item 15 is the recombinant MVA of item 13 or 14, wherein the nucleotide sequence encoding the RSV F membrane glycoprotein comprises a nucleotide sequence encoding the amino acid sequence of SEQ ID NO:2 or comprises the nucleotide sequence of SEQ ID NO:1. Item 16 is the recombinant MVA of any of items 8 to 13, wherein the nucleotide sequence encoding the RSV G membrane glycoprotein is from RSV strain A, preferably from strain A2, and/or B. Item 17 is the recombinant MVA of any of items 8 to 13 that comprises a nucleotide sequence encoding an RSV G membrane glycoprotein that comprises a nucleotide sequence encoding the amino acid sequence of SEQ ID NO:4 or comprising the nucleotide sequence nucleotide sequence of SEQ ID NO:3. Item 18 is the MVA of claim 8 or the recombinant MVA of any one of claims 8 to 17 wherein the recombinant MVA comprises or the MVA itself is MVA-BN® virus as deposited at the European Collection of Cell Cultures (ECACC) under number V00083008.

Item 19 is a vial comprising the pharmaceutical composition of item 6 or 7, wherein the pharmaceutical composition comprises at least 1×10⁸ IU/mL, at least 3×10⁸ IU/mL, at least 5×10⁸ IU/mL, or at least 7×10⁸ IU/mL of MVA or recombinant MVA.

Item 20 is a pharmaceutical composition comprising the MVA of item 8 or the recombinant MVA of any of items 8 to 18. Item 21 is a pharmaceutical composition comprising at least 1×10⁸ IU/mL, at least 3×10⁸ IU/mL, at least 5×10⁸ IU/mL, or at least 7×10⁸ IU/mL of the recombinant MVA of any of items 6 to 18. Item 22 is a vial comprising the pharmaceutical composition of item 20 or item 21, optionally a vaccine containing 100 ng or less of host cell DNA per vaccine dose.

Item 23 is the method of item 1, wherein said cell culture is a suspension quail cell culture infected with an MVA or recombinant MVA, wherein said population or cell culture comprises about 1×10⁶ to 1×10⁸ quail cells per mL and at least 1×10³ virus per mL of the MVA or recombinant MVA, or at least 1×10⁶, 1×10⁸, or 1×10⁹ MVA or recombinant MVA per mL. Item 24 is the method of item 1, wherein said cell culture is a suspension quail cell culture comprising MVA or recombinant MVA that has been cultured so that the cell culture or population comprises about 1×10⁶ cells/mL to 5×10⁸ cells/mL and about 1×10⁷ virus per mL to 1×10⁹ virus per mL. Item 25 is the method of item 1, wherein said cell culture is a suspension quail cell culture that comprises CCX.E10 quail cells.

Item 26 is a pharmaceutical composition comprising MVA-RSV or MVA-BN-RSV made by the process of any of items 1-5 for the prevention of Lower Respiratory Tract Disease caused by RSV. Item 27 is a pharmaceutical composition comprising an MVA or recombinant MVA made by the method of any of items 1-5, optionally a vaccine containing 100 ng or less of host cell DNA per vaccine dose.

### EXAMPLES

### Example 1: Purification of Recombinant MVA from Avian Cell Cultures

Each of MVA-BN-RSV and MVA-BN® virus were amplified (separately) in a suspension quail cell culture, then purified as follows. Material (cell culture) was collected into a mixer tank and treated with TrypLE (Gibco, Fisher Scientific, Waltham, MA, USA) for an hour at ambient temperature, followed by inactivation with trypsin inhibitor for 15 minutes. The pH of the material was adjusted to 8.5, MgCl₂ was added to 2 mM, and 20 U/mL Denarase® endonuclease added. NaCl was added to a final concentration of 1M, and the material was incubated overnight at 5-8° C. with stirring. Cells were lysed using high pressure homogenization (HPH), and cell debris and remaining cells were removed by depth filtration using a 5 μm depth filter. The material was then incubated at 30° C. overnight with stirring and filtered using cassette-based Tangential Flow Filtration (TFF) with a 0.1 μm cutoff PESU filter or a PS hollow fiber with a 0.05 μm cutoff and an inner diameter of 0.5 mm. In this TFF, ultrafiltration was performed and then diafiltration using DF1 buffer (10 mM Tris, 2 mM MgCl₂, pH 8.2), including a flush of the system at the end that was pooled with the retained material. A second enzymatic treatment was performed with Denarase® endonuclease at 100 U/mL with 2 mM MgCl₂ and incubated overnight at room temperature with stirring. A second TFF step was then performed using cassette-based TFF with a 0.1 μm cutoff PESU filter or PS hollow fiber with a 0.05 μm cutoff and an inner diameter of 0.5 mm. In this filtration, diafiltration was performed with DF2 buffer (10 mM Tris, 140 mM NaCl, pH 7.7); optionally, a second buffer exchange was performed.

In four exemplary runs, this process yielded approximately 65% recovery with 85 ng/dose host cell DNA and 1 mg/dose total protein. This result almost meets the criteria recommended by regulatory authorities for final concentration of host cell DNA in the final product of 10 ng or less per vaccine dose; also the process met the overall recovery of more than 50% in order to be economically feasible as a vaccine production process. In addition, the remaining host cell DNA was fragmented, making any viable coding nucleic acids in the vaccine less likely.

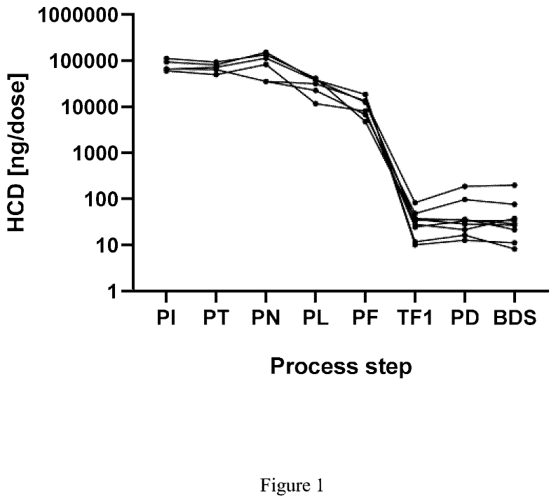

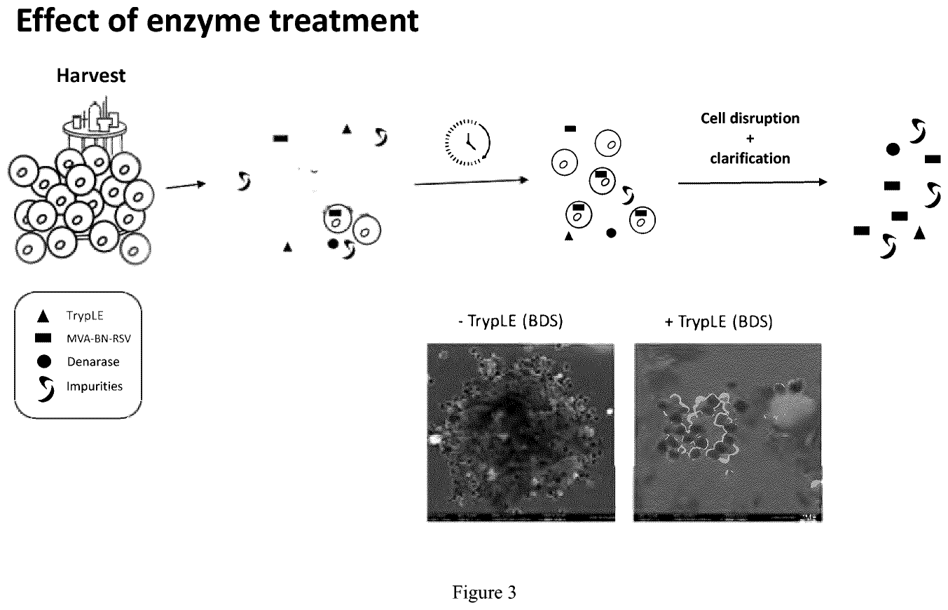

Among the surprising aspects of these experiments was that trypsin enzymatic activity had a positive effect in later process steps and on the end product, even though the trypsin activity was only present earlier in the process even before the cells were lysed. FIG. 1 shows the effects of Denarase® endonuclease on the amount of Host Cell DNA (“HCD”) per dose of vaccine at various stages of an exemplary downstream process. FIG. 2 shows that addition of NaCl during initial steps of the downstream process provides a surprising decrease in Host Cell DNA (HCD) per dose of vaccine at later stages of an exemplary downstream process even though at early stages of the process the amount of Host Cell DNA per dose of vaccine is increased. FIG. 3 includes a schematic showing the hypothesized effect of the first enzymatic treatment step with trypsin enzymatic activity (e.g., TrypLE) and nuclease (e.g., Denarase® enzyme) on the harvested cells, amplified virus, and impurities that result from lysis of the cells during the virus purification process. FIG. 3 also shows transmission electron micrographs of cells with and without trypsin enzymatic treatment, where the cells in the absence of trypsin treatment form a compact clump (left-hand picture) and cells treated with trypsin enzymatic treatment appear to be much more loosely associated in a smaller group (right-hand picture) in which each cell appears to be in contact with the surrounding environment.

Another surprising aspect of the methods of the invention is that Denarase® endonuclease was able to digest the majority of Host Cell DNA (HCD) at high NaCl concentrations of about 1M, far above the manufacturer's recommended levels of up to 150 mM NaCl, and above levels at which the manufacturer indicates this enzyme should be active.

A comparison was performed in parallel of the downstream purification process with and without adjusting the pH in the first enzymatic treatment step. The results were very similar. Specifically, in the absence of adjustment of the pH, the recovery as % of lysis was 52.9% for an obtained number of doses of 5341 doses/liter of bioreactor with HCD at 3.1 ng/dose, while in the standard process that includes pH adjustment, the recovery was 55.0% for an obtained number of doses of 5572 doses/liter of bioreactor with HCD at 1.3 ng/dose. Thus, in some embodiments, the adjustment of pH in the first enzymatic treatment may be omitted without adversely affecting the results.

### Example 2: Purification of MVA-BN from Avian Cell Cultures

MVA-BN® virus was amplified in a suspension quail cell culture, then Bulk Drug Substance (BDS) was purified as follows. This method is similar to that described in Example 1, but comprises only one Tangential Flow Filtration (TFF) step (in some instances, referred to as a “short process”).

Material (cell culture) was collected into a mixer tank and treated with TrypLE™ enzyme (Gibco™, Fisher Scientific™, Waltham, MA, USA) for an hour at ambient temperature, followed by inactivation with trypsin inhibitor for 15 minutes. The pH of the material was adjusted to 8.5, MgCl₂ was added to 2 mM, and 20 U/mL Denarase® endonuclease was added. NaCl was added to a final concentration of 1M, and the material was incubated for about 15 hours at 5-8° C. with stirring. Cells were lysed using high pressure homogenization (HPH) at 500 bar, and cell debris and remaining cells were removed by depth filtration using a 5 μm depth filter (sometimes referred to as “clarification”). The material was then incubated at room temperature overnight with stirring. The material was then filtered using Tangential Flow Filtration (TFF) with a PS hollow fiber with a 0.05 μm cutoff. Diafiltration was performed using DF2 buffer (10 mM Tris, 140 mM NaCl, pH 7.7), including a flush of the system at the end that was pooled with the retained material. An overall concentration of 4.5× was achieved. Similar results were obtained with this process when BB2 buffer was used for diafiltration.

Different runs of this process were performed on material (cell culture) collected from bioreactors comprising ~3 liter cultures and ~50 liter cultures and the results compared. In 11 runs from the 3-liter cultures, the average HCD in ng/dose was 1.3, while in a run from a 50-liter culture, the HCD in ng/dose was 5.3. Similar HCD (ng/dose) results were obtained from the “short process” in comparison to the “long process.” In these experiments, the “long process” further comprised an additional enzymatic treatment with Denarase® enzyme (100 U/mL) with 2 mM MgCl₂, a room temperature incubation for ~15 hours, and a second Tangential Flow Filtration (TFF). This second TFF used 0.1 micrometer cut-off PESU, 10× overall concentration, and 10× DF in total using a 2-step DF first with DF2 buffer 5×, then BB2 buffer 5× plus a flush to produce the final Buld Drug Substance (BDS). Because this “long process” produced approximately the same HCD in ng/dose as the “short process,” the “short process” was used for further production due to reduced cost of reagents and less time required to produce BDS. In this manner, the invention provides several processes that generate MVA-BN® Bulk Drug Substance with less than 10 ng Host Cell DNA per dose.

### Example 3: Purification of MVA-BN-RSV from Avian Cell Cultures

An exemplary MVA-BN-RSV is MVA-mBN294, a recombinant MVA that encodes an antigenic determinant of at least the RSV nucleocapsid N protein, the RSV M2 matrix protein, the RSV G membrane glycoprotein and the RSV F membrane glycoprotein (see, e.g., WO 2014019718, herein incorporated by reference in its entirety).

MVA-mBN294 was amplified in a suspension quail cell culture, then purified as follows. Material (cell culture) was collected into a mixer tank and treated with TrypLE™ enzyme (Gibco™, Fisher Scientific™, Waltham, MA, USA) for an hour at ambient temperature, followed by inactivation with trypsin inhibitor for 15 minutes. The pH of the material was adjusted to 8.5, MgCl₂ was added to 2 mM, and 20 U/mL Denarase® endonuclease was added. NaCl was added to a final concentration of 1M, and the material was incubated overnight at 5-8° C. with stirring. Cells were lysed using high pressure homogenization (HPH) at 500 bar, and cell debris and remaining cells were removed by depth filtration using a 5 μm depth filter (“clarification”). The material was then incubated at room temperature overnight with stirring and filtered using Tangential Flow Filtration (TFF) with a PS hollow fiber with a 0.05 μm cutoff. In this TFF, ultrafiltration was performed, yielding a concentration of 5.75×, then one round of diafiltration followed by a second ultrafiltration yielding a concentration of 7.65× and seven rounds of diafiltration, including a flush of the system at the end that was pooled with the retained material. All of these steps used DF1 buffer (10 mM Tris, 2 mM MgCl₂, pH 8.2).

A second enzymatic treatment was performed with Denarase® nuclease at 100 U/mL with 2 mM MgCl₂ and incubated for about 15 hours at room temperature with stirring. A second TFF step was then performed using PS hollow fibers with a 0.05 μm cutoff and an inner diameter of 0.5 mm. In this filtration, diafiltration was performed five timeswith DF2 buffer (10 mM Tris, 140 mM NaCl, pH 7.7) and then five times with BB2 buffer (10 mM Tris, 140 mM NaCl, pH 7.7, 10% w/w sucrose, 2% Sorbitol, 100 mM L-arginine), followed by a flush that was combined with the retained material to produce the final Bulk Drug Substance (“BDS”), providing a 10× increase in overall concentration.

This process was performed in 3 L bioreactors, in 50 L bioreactors, and in 200 L bioreactors. In five runs with 3 L bioreactors, the average HCD ng/dose was 61.6; in three runs in the 40 L bioreactors, the average HCD ng/dose was 72.3, and in two runs in the 200 L bioreactors, the average HCD/dose was 78.0. These results show that the process is scalable and can produce BDS with less than 100 ng/dose HCD.

### Example 4: Purification of MVA-BN-WEV from Avian Cell Cultures

MVA-BN-WEV is a recombinant MVA comprising nucleic acids encoding antigens of Eastern Equine Encephalitis Virus, Western Equine Encephalitis Virus, and Venezuelan Equine Encephalitis Virus, and is being developed for use as a vaccine against these viruses (see, e.g., MVA-mBN396B as described in WO 2017129765). MVA-BN-WEV was amplified in a suspension quail cell culture, then purified as follows. Material (cell culture) was collected into a mixer tank and treated with TrypLE™ enzyme (Gibco™, Fisher Scientific™, Waltham, MA, USA) for an hour at ambient temperature, followed by inactivation with trypsin inhibitor for 15 minutes. The pH of the material was adjusted to 8.5, MgCl₂ was added to 2 mM, and 20 U/mL Denarase® nuclease was added. NaCl was added to a final concentration of 1M, and the material was incubated for about 15 hours at 5-8° C. with stirring. Cells were lysed using high pressure homogenization (HPH) at about 500 bar, and cell debris and remaining cells were removed by depth filtration using a 5 μm depth filter (“clarification”). The material was then incubated at room temperature overnight with stirring and filtered using Tangential Flow Filtration (TFF) with a filter comprising PS hollow fiber with a 0.05 μm cutoff and an inner diameter of 0.5 mm. In this TFF, ultrafiltration was performed for an increase in concentration of 6×, then one round of diafiltration, followed by ultrafiltration for an increase of 8× and nine rounds of diafiltration including a flush of the system at the end that was pooled with the retained material. All of these steps used DF1 buffer (10 mM Tris, 2 mM MgCl₂, pH 8.2).

A second enzymatic treatment was performed with Denarase® nuclease at 100 U/mL with 2 mM MgCl₂ and incubated for about 15 hours at room temperature with stirring. A second TFF was then performed using PS hollow fiber with a 0.05 μm cutoff and an inner diameter of 0.5 mm. Diafiltration was performed ten times, followed by a flush, all with BB2 buffer to yield a 10.5× increase in overall concentration and the final Bulk Drug Substance (BDS).

This process was performed with material produced in 3 liter (“3L”) bioreactors, 50 liter (“50L”) bioreactors, and 200 liter (“200L”) bioreactors. In 7 runs using 3 L bioreactors, the average HCD in ng/dose was 16.3; in one run with a 50 L bioreactor, the HCD was 40.8 ng/dose, and in one run with a 200 L bioreactor, the HCD was 12.9 ng/dose. This data shows that the process is scalable and produces BDS suitable for commercial vaccines, with HCD in the range of 10 ng/dose.

These trials also showed that for MVA-BN-WEV the “short process,” which ends after one Tangential Flow Filtration, could provide similar results to the “long process,” which further includes a second Denarase® nuclease treatment and a second Tangential Flow Filtration. As the “short process” uses fewer reagents and requires less time, it provides additional benefits for commercial production.

### Example 5: Comparison of Quail Cell Processes to CEF Cell Process

Several rounds of experiments were conducted to compare downstream purification of virus from quail cells to the results obtained from CEF cells. MVA-BN® virus and recombinant MVAs were amplified in chicken embryonic fibroblast (CEF) cultures or in quail cell cultures, then purified in downstream processes of the instant invention. Cells were amplified, infected with virus, and cultured for viral amplification in wave bag cultures (for example, in a total volume of 300 liters (300 L)) or in bioreactors (e.g., a 250 liter (250 L) bioreactor). The viruses were then purified essentially as follows.

For wavebag cultures, cell material was collected, then sonicated and clarified using a Viafuge® centrifuge (CARR Biosystems®, Clearwater, FL, US). Tangential Flow Filtration was then performed using cartridges comprising 0.1 μm PES with ultrafiltration followed by 3× diafiltration. The material was then treated with Denarase® nuclease, followed by a second round of Tangential Flow Filtration that comprised ultrafiltration followed by 15× diafiltration.

For bioreactor cultures, CEF cells were grown in VP-SFM media (Thermo Fisher Scientific®, Waltham, MA, US) with Cytodex® microcarriers (Sigma Aldrich™, St. Louis, MO, USA). Following infection with virus and virus growth, the bioreactor culture was mixed at maximum speed (~145 rpm) for approximately 5 hours to release virus from the cells and the supernatant containing the virus was passed through a Harvestainer™ system (Thermo Fisher Scientific®, Waltham, MA, US) to remove the microcarriers. Media was added to the remaining bioreactor debris, mixed to release additional virus, and the collection process repeated. The pooled collected material was then sonicated and clarified by centrifugation using a Viafuge® centrifuge (CARR Biosystems®, Clearwater, FL, US). Tangential Flow Filtration was then performed using cartridges comprising 0.1 μm PES; the first round comprised ultrafiltration and 3× diafiltration. The material was then treated with 100 U/mL Denarase for about 3 hours with rocking at ambient temperature. A second round of Tangential Flow Filtration comprised ultrafiltration and 15× diafiltration. Material was then batch centrifuged at 10,800 rcf for 45 minutes at 4 degrees C.

Results of these processes using CEF cells were compared to those from the processes using quail cells described above (see Table 1). For these calculations, a dose was considered to be 1.58×10⁹ InfU/mL in a final (vaccine) volume of 0.5 mL.

TABLE 1
MVA-BN-RSV Downstream Processing
Results in CEF vs. Quail Cells

| | | CEF | Quail cell | |
|---|---|---|---|---|
| | CEF | 300 L | 50 and | Quail |
| | 250 L | (wavebag | 200 L | cell 3 L |
| | bioreactor | culture) | bioreactor | bioreactor |
| Value | (n = 12) | (n = 4) | (n = 2) | (n = 7) |
| Start titer | 7.16 | 7.69 | 8.46 | 8.61 |
| (log10 InfU/mL) | | | | |
| BDS titer | 9.33 | 9.37 | 9.63 | 9.66 |
| (log10 InfU/mL) | | | | |
| Recovery | 107.2% | 34.9% | 151.5% | 116.2% |
| (% relative to | | | | |
| vessel harvest) | | | | |
| # of doses in | 6,269 | 7,050 | 206,982 | 219,800 |
| theoretical 500 L | | | | |
| vessel | | | | |
| HCD (ng/dose) | 19,877.0 | 4,828.8 | 69.0 | 32.3 |

Thus, as shown in Table 1, the results of the downstream processes with quail cell cultures were unexpectedly superior to those with Chicken Embryonic Fibroblast (CEF) cells, providing much higher yields of doses per amount of cell culture with a much lower level of Host Cell DNA (HCD) per dose of MVA-BN-RSV.

TABLE 2
MVA-BN ® Downstream Processing
Results in CEF vs. Quail Cells

| | CEF 300 L | Quail cell | Quail cell |
|---|---|---|---|
| | (wavebag | 50 L | 3 L |
| | culture) | bioreactor | bioreactor |
| Value | (n = 10) | (n = 1) | (n = 8) |
| Start titer (log10 | 8.17 | 8.11 | 9.29 |
| InfU/mL) | | | |
| BDS titer (log10 | 8.93 | 9.37 | 10.03 |
| InfU/mL) | | | |
| Recovery (% relative to | 57.6% | 406.2% | 123.8% |
| vessel harvest) | | | |
| # of doses in theoretical | 112,079 | 693,627 | 3,196,455 |
| 500 L vessel | | | |
| HCD (ng/dose) | 1,823.3 | 5.3 | 1.2 |

As shown in Table 2 for MVA-BN®, the results of the downstream processes with quail cell cultures were unexpectedly superior to those with Chicken Embryonic Fibroblast (CEF) cells, providing much higher yields of doses per amount of cell culture with a much lower level of Host Cell DNA (HCD) per dose.

In Table 2, some of the recovery % relative to vessel harvest was above 100%. This likely reflects that much of the virus remains inside cells at the time of vessel harvest and is later released during the downstream purification process. For this reason, the post-lysis virus titer may be more useful as a point of comparison, as that has been shown to represent peak virus titer during the process.

TABLE 3
MVA-BN-WEV Downstream Processing
Results in CEF vs. Quail Cells

| | | Quail cell | |
|---|---|---|---|
| | CEF 200 L | 50 L or | Quail cell |
| | (wavebag | 200 L | 3 L |
| | culture) | bioreactor | bioreactor |
| Value | (n = 3) | (n = 2) | (n = 3) |
| Start titer (log10 | 7.76 | 8.49 | 8.54 |
| InfU/mL) | | | |
| BDS titer (log10 | 8.52 | 9.57 | 9.65 |
| InfU/mL) | | | |
| Recovery (% relative to | 48.3% | 123.8% | 134.9% |
| vessel harvest) | | | |
| # of doses in theoretical | 12,092 | 177,559 | 219,245 |
| 500 L vessel | | | |
| HCD (ng/dose) | 8,678.3 | 19.7 | 24.2 |

As shown in Table 3 for MVA-BN-WEV, the results of the downstream processes with quail cell cultures were unexpectedly superior to those with Chicken Embryonic Fibroblast (CEF) cells, providing much higher yields of doses per amount of cell culture with a much lower level of Host Cell DNA (HCD) per dose.

It is to be understood that both the foregoing general and detailed description are exemplary and explanatory only and do not restrict or limit the invention as claimed. The accompanying drawings, which are incorporated in and constitute a part of this specification, illustrate various embodiments of the invention and together with the description, serve to explain the principles of the invention.

We claim:

1. A method of producing a pharmaceutical composition from an avian cell culture infected with MVA or recombinant MVA, comprising the steps of:
(a) harvesting cell cultures comprising avian cells infected with MVA or recombinant MVA to produce collected material;
(b) treating the collected material with a protease having trypsin activity;
(c) treating the material with a nuclease
(d) adjusting pH and/or salt content of the material;
(e) lysing cells in the material;
(f) filtering the material to remove cell debris and remaining cells;
(g) continuing incubation with nuclease for a period of time;
(h) concentrating the product using tangential flow filtration; and
(i) suspending the product in a suitable buffer to provide a pharmaceutical composition.

2. The method of claim 1, further comprising after step (h) and before step (i) the steps of:
(A) treating the material with a nuclease; and
(B) concentrating the product using tangential flow filtration.

3. The method of claim 1 or 2 wherein said nuclease in step (c) and/or step (A) is Denarase® endonuclease.

4. The method of claim 3, further comprising the addition of NaCl to a final concentration of at least 0.5M or about 1 M during step (d).

5. The method of claim 1, wherein step (i) comprises the use of diafiltration to exchange the buffer in which the product is suspended.

6. The method of claim 1, wherein said avian cells are quail cells.

7. The method of claim 6, wherein said quail cells are CCX.E10 cells.

8. The method of claim 1 or 7, wherein said MVA is MVA-BN® virus.

9. The method of claim 1 or 7, wherein said recombinant MVA is MVA-BN-RSV.

10. A pharmaceutical composition comprising MVA-BN® virus or MVA-BN-RSV made by the method of any of claims 1-7.

11. A vaccine comprising the pharmaceutical composition of claim 10, wherein the amount of host cell DNA per vaccine dose is less than 100 ng.
